(19) United States
(12) Patent Application Publication
(10) Pub. No.: US 2007/0078480 A1
(43) Pub. Date: Apr. 5, 2007

(54) Self-expanding biodegradable or water-soluble vaso-occlusive devices

(75) Inventors: Bronislava Belenkaya, Campbell, CA (US); Elaine Lee, Sunnyvale, CA (US)

Correspondence Address: ROBINS & PASTERNAK, 1731 EMBARCADERO ROAD, SUITE 230, PALO ALTO, CA, 94303, US

(73) Assignee: Boston Scientific Scimed, Inc.

(21) Appl. No.: 11/242,981

(22) Filed: Oct. 4, 2005

(51) Int. Cl. A61M 29/00

(52) U.S. Cl. 606/200

(57) ABSTRACT

This is a device for occluding a space within the body. In particular, the device comprises a self-expanding biodegradable or water-soluble material that is placed within the space to be occluded.

### FIELD OF THE INVENTION

Compositions and methods for repair of aneurysms are described. In particular, vaso-occlusive devices comprising an expandable material are disclosed, as are methods of making and using these devices.

### BACKGROUND

An aneurysm is a dilation of a blood vessel that poses a risk to health from the potential for rupture, clotting, or dissecting. Rupture of an aneurysm in the brain causes stroke, and rupture of an aneurysm in the abdomen causes shock. Cerebral aneurysms are usually detected in patients as the result of a seizure or hemorrhage and can result in significant morbidity or mortality.

There are a variety of materials and devices which have been used for treatment of aneurysms, including platinum and stainless steel microcoils, polyvinyl alcohol sponges (Ivalone), and other mechanical devices. For example, vaso-occlusion devices are surgical implements or implants that are placed within the vasculature of the human body, typically via a catheter, either to block the flow of blood through a vessel making up that portion of the vasculature through the formation of an embolus or to form such an embolus within an aneurysm stemming from the vessel. One widely used vaso-occlusive device is a helical wire coil having windings that may be dimensioned to engage the walls of the vessels. (See, e.g., U.S. Pat. No. 4,994,069 to Ritchart et al.) Other less stiff helically coiled devices have been described, as well as those involving woven braids. See, e.g., U.S. Pat. No. 6,299,627.

U.S. Pat. No. 5,354,295 and its parent, U.S. Pat. No. 5,122,136, both to Guglielmi et al., describe an electrolytically detachable embolic device. Vaso-occlusive coils having little or no inherent secondary shape have also been described. For instance, co-owned U.S. Pat. Nos. 5,690,666; 5,826,587; and 6,458,119 by Berenstein et al., describes coils having little or no shape after introduction into the vascular space. U.S. Pat. No. 5,382,259 describes non-expanding braids covering a primary coil structure. Vaso-occlusive devices comprising one or more coatings have also been described. U.S. Pat. No. 6,280,457 discloses vaso-occlusive devices that include biodegradable coatings.

Still another approach to the embolization of an abnormal vascular site is the injection into the site of a hydrogel, such as poly (2-hydroxyethyl methacrylate) (“pHEMA” or “PHEMA”); or a polyvinyl alcohol foam (“PAF”). See, e.g., Horak et al., “Hydrogels in Endovascular Embolization. II. Clinical Use of Spherical Particles”, Biomaterials, Vol. 7, pp. 467-470 (November 1986); Rao et al., “Hydrolysed Microspheres from Cross-Linked Polymethyl Methacrylate”, J. Neuroradiol., Vol. 18, pp. 61-69 (1991); Latchaw et al., “Polyvinyl Foam Embolization of Vascular and Neoplastic Lesions of the Head, Neck, and Spine”, Radiology, Vol. 131, pp. 669-679 (June, 1979).

U.S. Pat. No. 5,258,042 to Mehta et al. describes stent devices for opening the interior of a vessel for blood flow that involve the use of hydrogel materials. However, this document indicates that such materials should not be placed inside aneurysms, for fear of rupture of the aneurysm from unlimited expansion of the hydrogel. These types of plugs or implants are primarily designed for obstructing blood flow through a tubular vessel or the neck of an aneurysm, and they are not easily adapted for precise implantation within an irregular-shaped vascular structure, such as an aneurysm, so as to fill substantially the entire volume of the structure.

Hydrogel materials have also been used to occlude aneurysms. U.S. Pat. No. 6,299,619 to Greene et al. describes an embolization device comprising one or more expansible, hydrophilic embolizing elements non-releasably carried on a filamentous carrier at spaced intervals along the length of the carrier, where the expansile elements expand upon application of saline. U.S. Pat. No. 6,723,108 to Jones et al. discloses an expansible hydrogel foam sleeve disposed around an elongated vaso-occlusive coil. However, the hydrogels described in these documents all are based on polymers or copolymers of a free radical polymerizable hydrophilic olefin monomer cross-linked with up to about 10% by weight of multiolefin-functional cross-linking agent. Residual monomers and cross-linking agents reside within these types of gels and must be exhaustively washed in an attempt to remove those residuals. Most wash procedures are ineffective at removing all non-cross-linked monomers and/or cross-linking agents and as a result, these agents or monomers can act as toxins or local inflammatory agents when implanted into a host. Therefore, hydrogels that incorporate a physical linkage may offer an advantage beyond those that require chemical cross-linking. In addition, non-vinyl monomers based polymers have definite advantages. It is surmised that integrity of physical hydrogel material is primarily derived from hydrogen bonding of different types along with ionic association, hydrophobic interacton, crosslinking by the crystalline segments and stereocomplex formation that assist in supporting polymer integrity (see, e.g., Tanaka et al. (1992) Macromolecules, 25: 1516-1523).

Furthermore, the hydrogels described by Greene et al also are non-biodegradable. That means that the material will persist within the body for the duration of the implant. However, oftentimes with aneurysms there are clinical symptoms due to the “mass” of the aneurysm (hereafter called mass effect). The mass effect can result in local blood flow constriction, parenchyma degradation and/or nerve compression. It is most often seen in aneurysms in the frontal lobe region whereby the ophthalmic nerve is compressed and vision impaired. Thus, a persistent material does not allow the aneurysm to shrink (due to tissue contracture) beyond the volume of the deposited material. With a degradable material, the material goes away over time and is replaced by host tissue. The degradation of the material may also allow further tissue contracture and aneurysm shrinkage, such that the clinical symptoms resulting from the mass effect also resolves.

Thus, there remains a need for vaso-occlusive devices comprising self-expanding, biodegradable or water-soluble materials that provide volumetric filling of the aneurysm and are gradually replaced by new tissue. These devices are placed into a body cavity in order to occlude the cavity and are of particular for aneurysms, including ruptured aneurysms.

### SUMMARY OF THE INVENTION

Thus, this invention includes novel occlusive compositions as well as methods of using and immobilizing these compositions.

In one aspect, the invention comprises a vaso-occlusive device for placement inside an aneurysm, the device comprising a self-expandable material that is physically cross-linked and, in addition, is degradable or water-soluble. In certain embodiments, the self-expandable material comprises a hydrogel material, for example, one or more hydrogels selected from either organic and/or inorganic (e.g., silicones, alumina, and ferric oxide) gels. Organic gels from which the hydrogel of the invention can be selected include, by way of example and not limited to, gels formed from polysaccharides and mucopolysaccharides including, hyaluronates, pectins, agarose and/or agar components, alginate, chitosan, chitosan derivatives such as chitosan modified with fructose, galactose and/or proteins such as collagen, gelatin and albumin; gels formed from carboxy alkyl celluloses, including but not limited to carboxymethyl cellulose; partially oxidized cellulose; gels formed from proteins (e.g., proteins that support cell growth and healing), including but not limited to fibronectin, gelatin, collagen, fibrin, poly or copolypeptides; gels formed from synthetic biodegradable polymers such polyphosphazenes, polyphosphoesters, polyanhydrides, polyethylene oxides, polyvinyl alcohols, polyethylene oxide-co-polypropyleneoxide block copolymers, copolymers of polylactides, polyglycolide, polycaprolactone, poly(3-hydroxy-butyric acid) with polyvinyl alcohol, PEGs, dextrans, alginic acids, sodium alginates and others such as described in U.S. Pat. No. 4,526,938 to Chirchill, et al.; gels formed from other hydroxy acids; and/or gels formed from other biologically degradable polymers that are non-toxic or are present as metabolites in the body.

In certain embodiments, the chemistry of the gels is regulated to have a finite and defined expansion capability, for example, by changing the PEO/PLA or PLA-PGA-PEO ratio and/or the length of the PLA-PEO blocks in the PEO-PLA or PLA-PGA-PEO block copolymers or by the density of physical cross-linking achieved by variation of Polymer/Physical cross-linker” such as Polymer/Cation ratio in Ca-alginate gels.

In another aspect, the invention includes any of the devices described herein in combination with an inner member. The inner member may be, for example, a vaso-occlusive coil, a stent, a filter, a suture, or any implantable device. In a preferred embodiment, the inner member comprises a vaso-occlusive coil having a linear primary configuration prior to deployment and a secondary three-dimensional configuration after deployment. The inner member may comprise, for example, a polymer or a metal (e.g., nickel, titanium, platinum, palladium, rhodium, gold, tungsten, iridium and alloys or combinations thereof such as nitinol or stainless steel). In certain embodiments, the self-expanding material at least partially surrounds the inner member at one or more locations, for example as a sheath or a sleeve disposed around the inner member.

In another aspect, the invention relates to devices in which a self-expanding material has been immobilized onto an inner member. In certain embodiments, the self-expanding material is coated, dipcoated or spray-dried on the inner member. In other embodiments, the self-expanding member is coated onto another material or device (e.g., degradable or nondegradable sutures), which combination is then attached to the inner member by any suitable means including, but not limited to, winding or braiding around the inner member and/or use of adhesives or fasteners.

In still further embodiments, the self-expanding material comprises one or more fibers, the fibers may be wound, braided or otherwise attached to the inner member directly. Alternatively, the fibers may be wound, braided or otherwise attached to another material or device (e.g., degradable or nondegradable suture material), which is then wound, braided or otherwise attached to the inner member. In certain embodiments, the fibers are nano- or micro- fibers.

Any of the devices described herein may further comprise a severable junction detachably which may be connected to a pusher element. In certain embodiments, the junction) is an electrolytically detachable assembly adapted to detach by imposition of a current; a mechanically detachable assembly adapted to detach by movement or pressure; a thermally detachable assembly adapted to detach by localized delivery of heat to the junction; a radiation detachable assembly adapted to detach by delivery of electromagnetic radiation to the junction or combinations thereof.

In another aspect, a method of occluding a body cavity is described, the method comprising introducing a vaso-occlusive device as described herein into the body cavity. In certain embodiments, the body cavity is an aneurysm.

These and other embodiments of the subject invention will readily occur to those of skill in the art in light of the disclosure herein.

### BRIEF DESCRIPTION OF THE FIGURES

FIG. 1 is an overview depicting an exemplary embodiment according to the present invention.

FIG. 2 depicts the exemplary device shown in FIG. 1 partially extruded from a deployment catheter.

It is to be understood that the drawings depict only exemplary embodiments and are not to be considered limiting in scope.

### DESCRIPTION OF THE INVENTION

Occlusive (e.g., embolic) compositions are described. The compositions described herein find use in vascular and neurovascular indications and are particularly useful in treating aneurysms, for example large diameter, curved or otherwise difficult to access vasculature, for example aneurysms, such as cerebral aneurysms. Methods of making and using these vaso-occlusive elements also form aspects of this invention.

All documents (publications, patents and patent applications) cited herein, whether above or below, are hereby incorporated by reference in their entireties.

It must be noted that, as used in this specification and the appended claims, the singular forms “a”, “an”, and “the” include plural referents unless the content clearly dictates otherwise. Thus, for example, reference to a device comprising “a self-expanding material” includes devices comprising of two or more materials.

Unlike previously described self-expanding materials used to treat aneurysms, the expandable materials described herein are not made with cross-linking agents and, in addition, are placed into the target cavity they are to occlude. Thus, the devices described herein comprise a biodegradable or water-soluble material that is deployed into the target body cavity (e.g., into an aneurysm), self-expands inside the body cavity and is gradually replaced by the new tissue

A number of self-expanding materials can be used in the devices described herein, including but not limited to hydrogel materials that are biodegradable or water-soluble. By “biodegradable” “degradable” or “bioabsorbable” is meant that the material is capable of being broken down especially into innocuous products over a period of time, ranging from days to weeks or to months or even years. By “water-soluble” is meant that the molecules of the material are capable of dissolving in water. Thus, biodegradable materials may include water-soluble biomaterials. By “hydrogel” is meant a material that absorbs a solvent (e.g. water), undergoes rapid swelling without discernible dissolution, and maintains three-dimensional networks capable of reversible deformation. Hydrogels currently used for implants are typically covalently (chemically) crosslinked networks of hydrophilic polymers, such as PEG, that form hydrogels (or aquagels) in the hydrated state. Although block copolymers having hydrophilic and hydrophobic regions, have been used to form hydrogels without chemical crosslinking agents, these polymers include hydrophobic and hydrophilic regions so that the hydrogel does not dissolve in water.

In a preferred embodiment, the hydrogel materials are not chemically cross-linked (although they may be physically cross-linked) and are biodegradable and/or water-soluble. The period of time it takes for the material to degrade and/or dissolve can range from hours (e.g., 1 to 24 hours or any time therebetween), to days (1 to 90 days or any day therebetween), to months (1 to 24 months of any time therebetween), or even to years.

The hydrogel of the present invention may include one or more polymer components, where the polymer is naturally occurring or synthetic, or a mixture of the foregoing. A hydrogel in accordance with the invention, may be formed, for example, from organic gels and inorganic gels.

Organic gels from which the hydrogel of the invention can be selected include, by way of example and not by way of limitation, gels formed from polysaccharides and mucopolysaccharides including, but not limited to hyaluronates, pectins, agarose, alginate; chitosan, chitosan derivatives such as chitosan modified with fructose, galactose and/or proteins such as collagen, gelatin and albumin; gels formed from proteins such as collagen, gelatin, fibronectin, fibrin, albumin, or poly or copolypeptides; carboxy alkyl celluloses, including but not limited to carboxymethyl cellulose; partially oxidized cellulose; and gels formed from synthetic biodegradable polymers such polyphosphazenes, polyphosphoesters, polyanhydrides, polyethylene oxides, polyethylene oxide-co-polypropyleneoxide block copolymers, polylactides, polyglycolide, polycaprolactone, poly(3-hydroxy-butyric acid), polyvinyl alcohols, PEG, dextran, alginic acid and sodium alginate and others such as described in U.S. Pat. No. 4,526,938 to Chirchill, et al.; gels formed from other hydroxy acids; and/or gels formed from other biologically degradable polymers that are non-toxic or are present as metabolites in the body.

The biodegradable self-expanding materials (e.g., gels) may have a regulated expansion that is regulated by gel polymer composition such as the PEO/PLA or PLA-PGA-PEO ratio and/or the length of the PLA -PEO blocks in the PEO -PLA or PLA-PGA-PEO block copolymers (see, e.g., Younes et al. (1987) J. Biomed. Mater Res 21(11):1301-1306;) Younes et al. (1988) Biomater Artif Cells Artif Organs. 16(4):705-19) or by the density of physical cross-linking achieved by variation of Polymer/Physical cross-linker such as Polymer/Ca ion ratio in Ca-alginate gels.

Inorganic gels from which the hydrogel of the invention can be selected include, by way of example and not by way of limitation, silicones, alumina, and ferric oxide.

The self-expanding vaso-occlusive materials may be shaped into a variety of forms prior to delivery, including, but not limited to, coils, cylinders, ovals, spheres, balloon-shapes, braids, etc.

The expanding materials described herein are advantageously used in combination with other vaso-occlusive devices, for example the GDC-type vaso-occlusive coils described above (see, e.g., U.S. Pat. Nos. 6,723,112; 6,663,607; 6,602,269; 6,544,163; 6,287,318; 6,280,457 and 5,749,894). Preferably, the self-expanding materials surround the additional vaso-occlusive device. The self-expanding material may be immobilized onto the surface of the vaso-occlusive device in the shape of a sleeve or as a sheath, or self-expandable fibers, or a self-expandable braid made from expandable fibers. Expandable material may also be used as a coating for non-swellable degradable or nondegradable sutures that surround the inner part of vaso-occlusive device. The self-expandable material may also be immobilized onto the surface of the inner member in the form of micro/nanofibers using nanotechnology methods. The self-expanding material may have one or more coatings or additives designed to slow or delay the expansion of the self-expanding material, including, by way of example only, biodegradable non-swelling polymers (PLGA, PLLA, PLCL or mixtures thereof) and/or low molecular weight additives such as sucrose, inorganic salts or alcohols (see, e.g., Otake et al, (1990) Macromolecules, 23:283-289).

Depicted in the Figures are exemplary embodiments of the present invention. Although the optional inner member is depicted as a coil, it will be appreciated that this is for purposes of illustration only and that the inner member can be of other shapes, for example different shaped coils, stents, mandrels, wires, filters or the like.

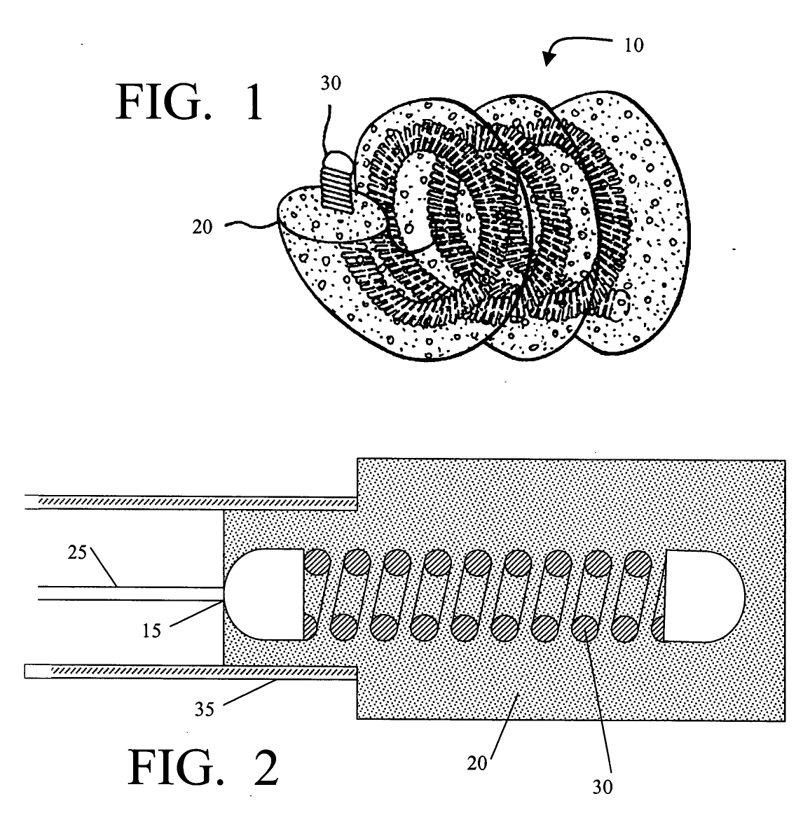

FIG. 1 depicts an exemplary embodiment of the inventive vaso-occlusive devices described herein. The device as a whole is generally designated (10) and is shown in a relaxed three-dimensional configuration. The self-expanding material (20) surrounds a helical shaped coil (30).

As noted above, the devices described herein or one or more of the components of these devices (e.g., inner member, self-expanding material) described herein may assume a variety of configurations including, but not limited to, braids, coil, stents (e.g., self-expanding stents) and combinations of these. Preferably, the devices (e.g., an inner coil component) are deployed in a primary linear configuration and assume a three-dimensional configuration upon deployment. For example, the devices may form a coil configuration or may have a substantially random space-filling relaxed configuration upon deployment. Thus, although depicted in the Figures as a coil (e.g., platinum coil), the inner member may be of a variety of shapes or configuration including, but not limited to, braids, wires, knits, woven structures, tubes (e.g., perforated or slotted tubes), injection-molded devices and the like. See, e.g., U.S. Pat. No. 6,533,801 and International Patent Publication WO 02/096273.

In certain embodiments, the inner member is a braided structure comprising one or more metals or metal alloys, for example, Platinum Group metals, especially platinum, rhodium, palladium, rhenium, as well as tungsten, gold, silver, tantalum, stainless steel and alloys of these metals. Preferably, the inner member comprises a material that maintains its shape despite being subjected to high stress, for example, “super-elastic alloys” such as nickel/titanium alloys (48-58 atomic % nickel and optionally containing modest amounts of iron); copper/zinc alloys (38-42 weight % zinc); copper/zinc alloys containing 1-10 weight % of beryllium, silicon, tin, aluminum, or gallium; or nickel/aluminum alloys (36-38 atomic % aluminum). Particularly preferred are the alloys described in U.S. Pat. Nos. 3,174,851; 3,351,463; and 3,753,700. Especially preferred is the titanium/nickel alloy known as “nitinol.” The inner member may also comprise a shape memory polymer such as those described in International Publication WO 03/51444.

In certain preferred embodiments, the inner member is a platinum coil. The inner member may also change shape upon release from the restraining member, for example change from a constrained linear form to a relaxed, three-dimensional configuration upon deployment.

FIG. 2 depicts a cross-section view of the device shown in FIG. 1 during deployment from a catheter (35). Inner coil (30) is surrounded by self-expanding material (20). Self-expanding material (20) expands in a self-regulated manner after extrusion from the catheter (35). The self-expanding material (20) may expand immediately upon extrusion from the catheter (35), as shown in FIG. 2, or it may expand in a more gradual or delayed manner.

Further, as shown in FIG. 2, any of the devices described herein may further comprise a detachment junction (15), which is severable. The detachment junction (15) may be connected to a pusher element, such as a pusher wire (25). The detachment junction can be positioned anywhere on the device, for example at one or both ends of the optional inner member (30). In certain embodiments, the inner member may be removed after deployment.

The severable junction(s) may be detached in a variety of ways, for example using an electrolytically detachable assembly adapted to detach by imposition of a current; a mechanically detachable assembly adapted to detach by movement or pressure; a thermally detachable assembly adapted to detach by localized delivery of heat to the junction; a radiation detachable assembly adapted to detach by delivery of electromagnetic radiation to the junction or combinations thereof. Furthermore, the detachment mechanism may be hydraulic, for example the pusher wire may be cannulated, for example to allow for saline injection through the pusher wire to push off the coil.

The devices described herein may also comprise additional components, such as co-solvents, plasticizers, coalescing solvents, bioactive agents, antimicrobial agents, thrombogenic agents, antithrombogenic agents (e.g., heparin), thrombus-stabilizing agents, antibiotics, pigments, radiopacifiers and/or ion conductors which may be coated using any suitable method or may be incorporated into the element(s) during production. See, e.g., co-owned U.S. patent application Ser. No. 10/745,911, U.S. Pat. No. 6,585,754 and WO 02/051460, incorporated by reference in their entireties herein. The bioactive materials can be coated onto the device (e.g., inner coil member) and/or can be placed in the vessel prior to, concurrently or after placement of one or more devices as described herein. For example, in embodiments in which the inner member is removed after deployment of the non-degradable device, one or more bioactive materials can be delivered to the vessel.

As noted elsewhere, the location of the device is preferably visible using fluoroscopy. A highly preferred method is to ensure that at least some of the elements (e.g., small pore non-degradable member and/or inner member) making up the device are provided with significant radio-visibility via the placement of a radio-opaque covering on these elements. The hydrogel may be imbibed with a radio-opaque agent to help confer radiopacity and allow visualization during embolization. A metallic coating of a metal having comparatively more visibility, during fluoroscopic use, than stainless steel is preferred. Such metals are well known but include gold and members of the Platinum Group described above.

One of more of the elements may also be secured to each other at one or more locations. For example, to the extent that various elements are thermoplastic, they may be melted or fused to other elements of the devices. Alternatively, they may be glued or otherwise fastened. Furthermore, the various elements may be secured to each other in one or more locations.

Methods of Use

The devices described herein are often introduced into a selected site using the procedure outlined below. This procedure may be used in treating a variety of maladies. For instance in the treatment of an aneurysm, the aneurysm itself will be filled (partially or fully) with the compositions described herein.

Conventional catheter insertion and navigational techniques involving guidewires or flow-directed devices may be used to access the site with a catheter. The mechanism will be such as to be capable of being advanced entirely through the catheter to place vaso-occlusive device at the target site but yet with a sufficient portion of the distal end of the delivery mechanism protruding from the distal end of the catheter to enable detachment of the implantable vaso-occlusive device. For use in peripheral or neural surgeries, the delivery mechanism will normally be about 100-200 cm in length, more normally 130-180 cm in length. The diameter of the delivery mechanism is usually in the range of 0.25 to about 0.90 mm. Briefly, occlusive devices (and/or additional components) described herein are typically loaded into a carrier for introduction into the delivery catheter and introduced to the chosen site using the procedure outlined below. This procedure may be used in treating a variety of maladies. For instance, in treatment of an aneurysm, the aneurysm itself may be filled with the embolics (e.g. vaso-occlusive members and/or liquid embolics and bioactive materials) which cause formation of an emboli and, at some later time, is at least partially replaced by neovascularized collagenous material formed around the implanted vaso-occlusive devices.

A selected site is reached through the vascular system using a collection of specifically chosen catheters and/or guide wires. It is clear that should the site be in a remote site, e.g., in the brain, methods of reaching this site are somewhat limited. One widely accepted procedure is found in U.S. Pat. No. 4,994,069 to Ritchart, et al. It utilizes a fine endovascular catheter such as is found in U.S. Pat. No. 4,739,768, to Engelson. First of all, a large catheter is introduced through an entry site in the vasculature. Typically, this would be through a femoral artery in the groin. Other entry sites sometimes chosen are found in the neck and are in general well known by physicians who practice this type of medicine. Once the introducer is in place, a guiding catheter is then used to provide a safe passageway from the entry site to a region near the site to be treated. For instance, in treating a site in the human brain, a guiding catheter would be chosen which would extend from the entry site at the femoral artery, up through the large arteries extending to the heart, around the heart through the aortic arch, and downstream through one of the arteries extending from the upper side of the aorta. A guidewire and neurovascular catheter such as that described in the Engelson patent are then placed through the guiding catheter. Once the distal end of the catheter is positioned at the site, often by locating its distal end through the use of radiopaque marker material and fluoroscopy, the catheter is cleared. For instance, if a guidewire has been used to position the catheter, it is withdrawn from the catheter and then the assembly, for example including the vaso-occlusive device at the distal end, is advanced through the catheter.

Once the selected site has been reached, the vaso-occlusive device is extruded, for example by loading onto a pusher wire. After extrusion, the self-expanding material expands in a controlled manner to final, predictable expanded volume (FIG. 2). The self-expanding material may expand immediately upon extrusion from the catheter, as shown in FIG. 2, or it may expand in a more gradual or delayed manner. Preferably, the vaso-occlusive device is loaded onto the pusher wire via a mechanically or electrolytically cleavable junction (e.g., a GDC-type junction that can be severed by application of heat, electrolysis, electrodynamic activation or other means). Additionally, the vaso-occlusive device can be designed to include multiple detachment points, as described in co-owned U.S. Pat. No. 6,623,493 and 6,533,801 and International Patent publication WO 02/45596. They are held in place by gravity, shape, size, volume, magnetic field or combinations thereof.

It will also be apparent that the operator can remove or reposition (distally or proximally) the device. For instance, the operator may choose to insert a device as described herein, before detachment, move the pusher wire to place the device in the desired location.

Modifications of the procedure and vaso-occlusive devices described above, and the methods of using them in keeping with this invention will be apparent to those having skill in this mechanical and surgical art. These variations are intended to be within the scope of the claims that follow.

What is claimed is:

1. A vaso-occlusive device for placement inside an aneurysm, the device comprising a self-expandable material that is noncovalently cross-linked and degradable or water-soluble.

2. The device of claim 1, wherein the self-expanding material comprises a hydrogel.

3. The device of claim 2, wherein the hydrogel comprises a polysaccharide or mucopolysaccharide.

4. The device of claim 2, wherein the hydrogel comprises a protein.

5. The device of claim 2, wherein the hydrogel comprises carboxy alkyl cellulose.

6. The device of claim 2, wherein the hydrogel comprises a synthetic polymer.

7. The device of claim 1, further comprising an inner member.

8. The device of claim 7, wherein the device has a linear primary configuration prior to deployment and a secondary three-dimensional configuration after deployment.

9. The device of claim 7, wherein the self-expanding material surrounds the inner member at one or more locations.

10. The device of claim 7, wherein the inner member comprises a metal.

11. The device of claim 10, wherein the metal is selected from the group consisting of nickel, titanium, platinum, palladium, rhodium, gold, tungsten, iridium, stainless steel and alloys or combinations thereof.

12. The device of claim 11, wherein the metal is nitinol.

13. The device of claim 7, wherein the self-expanding material is a sheath or a sleeve disposed around the inner member.

14. The device of claim 7, wherein the self-expanding material is coated, dipcoated or spray-dried on the inner member.

15. The device of claim 7, wherein the self-expanding material is coated onto a degradable or nondegradable polymeric or metallic suture, and wherein the sutures are attached to the inner member.

16. The device of claim 15, wherein the sutures are wound or braided around the inner member.

17. The device of claim 7, wherein the self-expanding material comprises one or more fibers.

18. The device of claim 17, wherein the fibers are wound or braided around the inner member.

19. The device of claim 17, wherein the fibers are wound or braided onto a degradable or nondegradable suture, and wherein the sutures are attached to the inner member.

20. The device of claim 17, wherein the fibers are nano or micro fibers.

21. The device of claim 7, wherein the inner member further comprises a detachment junction.

22. The device of claim 21, wherein the detachment junction comprises an electrolytically detachable end adapted to detach from a pusher by imposition of a current on the pusher.

23. The device of claim 1, further comprising an additional component.

24. The device of claim 23, wherein the additional component is bioactive.

25. A method of occluding a body cavity comprising introducing a vaso-occlusive device according to claim 1 into the body cavity.

26. The method of claim 25, wherein the body cavity is an aneurysm.

27. The method of claim 26, wherein the aneurysm is ruptured.

28. The device of claim 23, wherein the additional component causes a delay or deceleration of the expansion of the self-expanding material.
